(19) United States
(12) Patent Application Publication
(10) Pub. No.: US 2009/0210029 A1
(43) Pub. Date: Aug. 20, 2009

(54) DEVICE AND METHOD TO POSITION A CANNULA FOR NERVE BLOCK

(75) Inventor: Ban C.H. TSUI, Edmonton (CA)

Correspondence Address: AKERMAN SENTERFITT, P.O. BOX 3188, WEST PALM BEACH, FL, 33402-3188, US

(73) Assignee: Pajunk GmbH & Co. KG Besitzverwaltung, Geisingen (DE)

(21) Appl. No.: 12/369,044

(22) Filed: Feb. 11, 2009

(30) Foreign Application Priority Data
Feb. 14, 2008 (DE) 20 2008 002 105.7

(51) Int. Cl. A61N 1/34; A61M 25/01

(52) U.S. Cl. 607/46; 604/21

(57) ABSTRACT

A device and method to position a cannula or a catheter for nerve block are provided for use with a unipolar cannula (10), which is electrically insulated around its axial length. The device includes a stimulation device (20) to generate an electrical signal, an electrically conductive bare stimulation electrode (12) provided on a distal end of the unipolar cannula (10) and a skin electrode (14), which can be applied to a body surface (26) of a patient with an electrically conductive contact. The stimulation electrode (12) and the skin electrode (14) can be connected to the stimulation device (20), so that the electrical signals are routed from the stimulation electrode (12) through a patient's body (28) to the skin electrode (14). Further, the stimulation device (20) includes a measuring device (22) to measure the electrical resistance between the stimulation electrode (12) and the skin electrode (14) and the stimulation device (20) includes an alarm device (24) which produces an audible alarm signal when the measured resistance exceeds an adjustable threshold value.

### TECHNICAL FIELD

The application relates to a device and a method to position a cannula for nerve block.

### BACKGROUND

For surgical operations on the lower and upper extremities a nerve block is frequently used, in particular a peripheral nerve block. An anesthetic is injected directly into the nerves supplying the extremities in question. In operations on the upper extremities, the anesthetic is injected, for example, into the nerve sheath surrounding the brachial plexus. In operation on the lower extremities, preferably the anesthetic in the epidural area is injected for epidural anesthesia, to introduce the anesthetic a cannula is used which is inserted through the patient's skin into the nerve sheath and the epidural area. If necessary, a catheter can be introduced through this cannula. The catheter can be used to introduce the anesthetic. The catheter can also be used to introduce the anesthetic into a position located at a distance from the catheter puncture point. Again, the catheter can remain in its position after removing the cannula to be able to introduce anesthetic for a longer period of time.

It is important for a nerve block that the distal point of the cannula or catheter is positioned in the most exact manner possible. On the one hand, the point should be positioned as close to the nerve as possible to apply the anesthetic as close to the nerve as possible in order to achieve an effective nerve block. On the other, damage to the nerves by the cannula or the catheter should be avoided.

It is known that unipolar nerve stimulation is needed to position a cannula (e.g. EP 0 966 922 B1) or a catheter (e.g. DE 198 07 487 C2). In this method, a bare electrically conductive stimulation electrode is provided on the distal point of the electrically insulated cannula or of the electrically insulated catheter, while an electrically conductive second skin electrode is provided on the patient's body surface. Using a stimulation device electrical pulses are fed to the stimulation electrode to trigger nerve reflexes. These nerve reflexes make it possible to identify the position of the distal point with the stimulation electrode. In this method of unipolar eletrostimulation it is easy to localize the position of the distal point of the cannula or catheter on the nerve. However, use of this method requires experience and skill on the part of the anesthetist to minimize the risk of damaging the nerve.

### SUMMARY

The task of the inventive embodiments is to improve the device and method for positioning a cannula or a catheter for nerve block in such a way that the positioning can be done precisely and with a minimum risk of nerve damage.

To solve this task according to the inventive embodiments, a unipolar cannula or a unipolar catheter is used with a bare electrically conductive stimulation electrode on the distal end and a skin electrode which can be applied to the patient's body surface. The stimulation electrode and the skin electrode are connected to a stimulation device which generated electrical signals. These electrical signals are conducted through the stimulation electrode to the patient's body tissue to the skin electrode. In the stimulation device the electrical resistance (impedance) of the body tissue is measured between the stimulation electrode and the skin electrode, i.e. the resistance of the current path which the electrical signals run through the tissue.

This impedance is virtually constant, while the point of the cannula is pierced through the skin and the muscle tissue of the patient. Also when the cannula point penetrates the nerve sheath or the epidural area of the spinal column and, possibly, the nerve sheath (epineurium) and the spinal cord is contacted from the outside, there is no significant change in impedance. If, however, the distal point of the cannula or catheter is inserted into the nerve sheath or the spinal cord, impedance quickly jumps because the nerve sheath has a different electrical conductivity and therefore different impedance. The different impedance of the nerve sheath therefore plays a crucial role in the overall impedance of the current path between the stimulation electrode and the skin electrode. The value of this method lies in the detection of impedance changes which signify intraneural needle placement. Depending on the species and the characteristic of impulse applied (e. g. frequency and pulse width), the noted change in impedance may be an increase or a decrease and it may even be transient in nature.

The stimulation device displays the electrical resistance measured between the stimulation electrode and the skin electrode. If the resistance change suddenly exceeds a set threshold value, this indicates that the point of the cannula or catheter has penetrated the nerve sheath. The stimulation device generates an optical or preferably acoustic alarm signal to acoustically display this penetration of the nerve sheath. The anesthesiologist can then withdraw the cannula or catheter until its distal point is removed from the nerve sheath. The above reliably avoids mechanical damage to the nerve by the cannula or catheter. In particular, it also avoids the chemical damage resulting from injection of an anesthetic into the nerve by the cannula or catheter.

During penetration by the cannula, frequently fluid is injected through the cannula to widen the tissue in front of the cannula point and thereby to make it easier to remove the catheter from the cannula point. To date a physiological common salt solution has been used. Since this common salt solution is electrically conductive, it distorts the impedance measurement. In particular, the conductive common salt solution can counteract the impedance increase when the cannula point penetrates the nerve sheath so that this penetration of the cannula point cannot be recognized immediately.

Therefore, in one embodiment, during the penetration of the cannula a physiological fluid is injected with a low electrical conductivity, e.g. a glucose-water solution, in particular D5W, that is, a solution of water and 5% dextrose. D5W has low conductivity, so that the impedance measurement is not influenced or influenced to a very minor degree.

Injection of D5W during the penetration of the cannula also provides an additional possibility for localizing the cannula point, based on the low electrical conductivity of D5W.

If the distal cannula point with the stimulation electrode is outside the nerve, e.g. in the subcutaneous fatty tissue, in the muscle tissue or also in the nerve sheath or in the epidural area, the displayed impedance has a basic value of approx. 8 to 12 kΩ. If D5W if injected the impedance initially increases due to the poor conductivity of D5W, though it quickly drops back to the base value because D5W is distributed in the tissue fluid.

If the cannula point accidentally penetrates a vessel, in particular a blood vessel, this does not result in a significant change in impedance because the intravascular fluid, e.g. blood, has basically the same conductivity as the tissue fluid. If additional D5W is injected, there is a brief increase in impedance but it returns very quickly to the normal value because the injected D5W is taken into the blood stream and the stimulation electrode is freely rinsed.

If the cannula point accidentally penetrates the intrathecal area, i.e. in the dura mater, the impedance decreases somewhat to about 4 to 6 kΩ, which is due to the fact that the cerebrospinal fluid (CSF) in the intrathecal area shows a high conductivity. If D5W is injected, there is an initial significant increase in impedance. The impedance returns to the original low base value due to the fact that D5W is distributed in the cerebrospinal fluid. This decrease to the original value is however slower than for intravascular injection because the CSF does not flow and therefore does not rinse the DSW.

### BRIEF DESCRIPTION OF THE FIGURES

An example of the inventive embodiments is described in greater detail below using a figure. It shows:

FIG. 1 schematically illustrates the device to position a cannula for nerve block,

FIG. 2 provides a diagram of the impedance value when positioning the cannula point on a nerve,

FIG. 3 illustrates the impedance time path for the injection of D5W outside a nerve,

FIG. 4 illustrates the impedance time path for intravascular injection of D5W, and

FIG. 5 illustrates the impedance time path for intrathecal injection of D5W

### DETAILED DESCRIPTION

The inventive device is shown schematically in an embodiment. The device comprises a hollow cannula 10 which is electrically insulated on its outer mantel surface. Preferably, cannula 10 is a steel cannula whose outer surface has an electrically insulated coating. On its distal point, cannula 10 contains an electrically conductive small exposed simulation electrode 12. For example, the stimulation electrode 12 can be an uncoated area of the metal cannula 10.

The device also comprises a skin electrode 14. The stimulation electrode 12 of the cannula 10 can be connected to the stimulation device 20 via a connector cable 16 and the skin electrode 14 connected via a connector cable 18.

The stimulation device 20 generates electrical signals, in particular electric pulses, which preferably have adjustable amplitude, pulse length and pulse frequency. The stimulation device 20 also contains a measuring device 22 which measures the electrical resistance (impedance) between the stimulation electrode 12 and the skin electrode 14. Finally, the stimulation device 20 comprises an alarm device 24 which produces an audible alarm signal when the impedance measured by measuring device 22 exceeds an adjustable threshold value. The alarm signal can be an optical signal, e.g. a warning light or a blinking light. Preferably, the alarm signal is an acoustic signal, so that this alarm signal is also recognized when the stimulation device 20 is not observed.

To position cannula 10 for a nerve block, the connector cables 16 and 18 are connected to the stimulation device 20. The skin electrode 14 is applied with an electrically conductive contact to the skin surface 26 of the patient's body 28, in a preferably electrically conductive fashion. Then the cannula 10 with its distal end equipped with the stimulation electrode 12 penetrated through the skin 26 into the patient's body 28 to position the distal point of the cannula in the proximity of a nerve 30 to be blocked. If the cannula point 30 is positioned on the nerve 30, an anesthetic will be fed through the cannula 10.

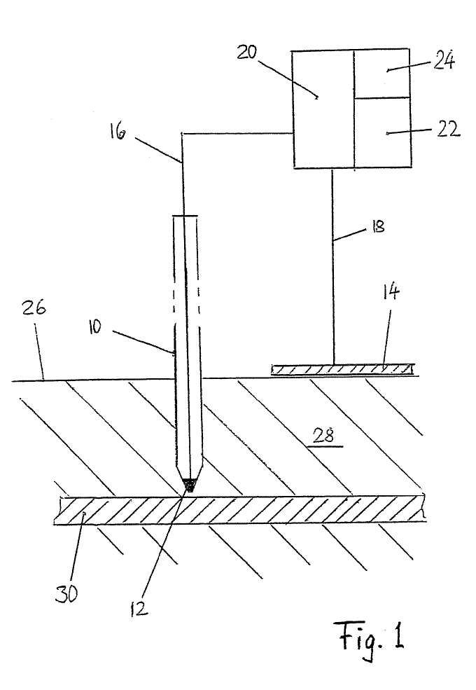

In FIG. 1 the cannula 10 and the nerve 30 are represented in a purely schematic fashion: The cannula 10 can be a known unipolar cannula as described for example in EP 0 966 922 B1. A catheter can if necessary be introduced through the cannula 10 as is known for example in DE 198 07 487 C2. If such a catheter is fed through the cannula 10, it can be designed as a unipolar catheter with a distal stimulation electrode as described in DE 198 07 487 C2. In this case, after the placing of the cannula 10, the stimulation electrode of the catheter can be connected via a connector cable 16 to the stimulation device 20, so that the impedance is measured and displaced between the stimulation electrode of the catheter and the skin electrode 14.

For the peripheral nerve block of the upper extremities the distal point of the cannula 10 is preferably introduced into the nerve sheath of the brachial nerve, whereby in particular also a catheter inside the nerve sheath can be extended to position the distal point of the catheter for the application of the anesthetic. For an epidural anesthesia, the cannula 10 penetrates into the epidural area and positioned in the dura mater. Here too the cannula 10 can be used to introduce a unipolar stimulation catheter which is extended into the epidural area in order to position its distal end for the application of the anesthetic.

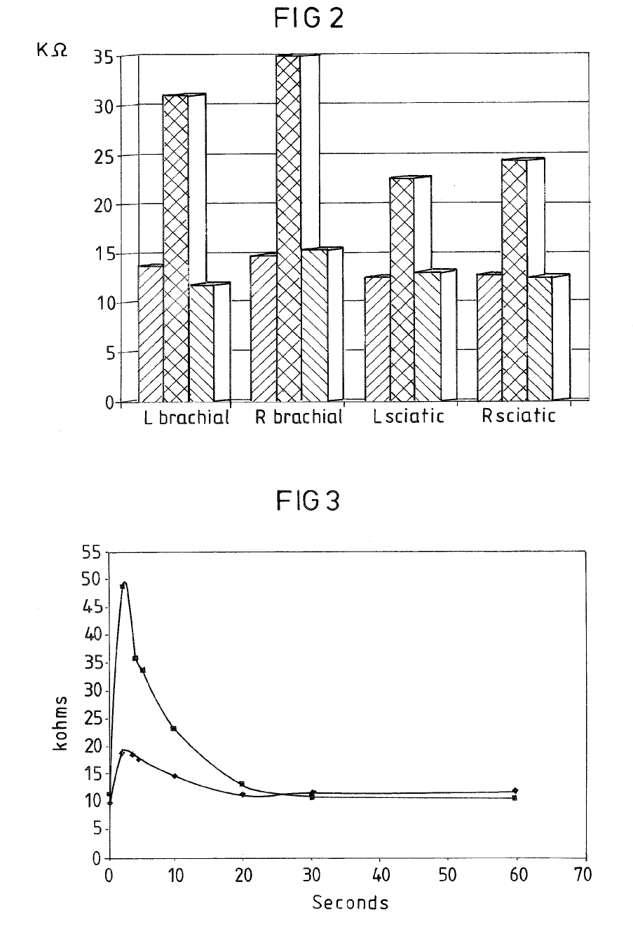

FIG. 2 shows in a diagram the measured impedance of the circuit measured by measuring device 22, which is a circuit created by the connector cable 16, the stimulation electrode 12, the current path through the patient's body 28, the skin electrode 14 and the connector cable 18. FIG. 2 shows examples of the positioning of the cannula 10 on the brachial nerve of the left arm and the right arm and on the sciatic nerve of the left leg and the right leg.

All impedance values presented are based on available data. However, these findings require cautions interpretation. Because of the small number of animal and/or patients studied to date, the milliamperage current settings are intended as guidelines and may require adjustment as experience increases. If the cannula 10 is pierced through the skin 26 into the patient's body 28, an impedance is measured which is represented respectively by the left white column. This impedance has a magnitude of 8 to 15 kΩ. Within these borders impedance varies to a small degree while the distal point of the cannula 10 penetrates the subcutaneous tissue and the muscle tissue up to the sheath of the nerve 30. If, however, the point of the cannula 10 penetrates into the nerve sheath with the stimulation electrode 12, due to the poor conductivity of the nerve sheath the measured impedance suddenly jumps to values that are distinctly over 20 kΩ. These impedance values are shown in FIG. 2 by the middle black column. Therefore, the threshold value of the impedance in the stimulation device 20 is set at about 20 kΩ. When the cannula point penetrates the nerve sheath therefore this threshold value is clearly exceeded so that an alarm signal is triggered by the alarm device 24. Due to this alarm signal the anesthesiologist knows that the cannula point has penetrated the nerve sheath accidentally and unwanted. He therefore withdraws the cannula 10 to pull the distal point of the cannula 10 with the stimulation electrode 12 from the nerve sheath. If the stimulation electrode 12 is withdrawn from the nerve sheath, the impedance again falls to the basic value between 8 and 15 kΩ, as shown in FIG. 2 by the right gray column. The anesthesiologist uses this to recognize that the distal point of the cannula is again located outside the nerve sheath and therefore in the desired position in which the anesthetic can be injected or a catheter inserted.

After the puncturing of the cannula 10, a physiological fluid with low conductivity is preferably injected to dilate the tissue in front of the cannula point so that the flexible catheter introduced by the cannula can more easily protrude from the distal cannula point. D5W is preferably used as such a fluid, i.e. a solution of 5% dextrose in water.

FIG. 3 shows examples of the time path of the measured impedance after the injection of D5W, whereby the distal point of the cannula 10 is located near the nerve sheath, though outside the nerve sheath. As FIG. 3 shows in two measurement curves, without injecting D5W a base value of the impedance is measured of ca. 10 to 15 kΩ. If at time 0, D5W is injected, initially D5W surrounds the stimulation electrode 12 with its low conductivity. Accordingly, the measured impedance rises sharply to values from 20 to 50 kΩ. As the D5W is distributed and dissolved in the tissue fluid of body 28, the impedance then falls in a period of 10 to 20 seconds to the original base value of 10 to 15 kΩ.

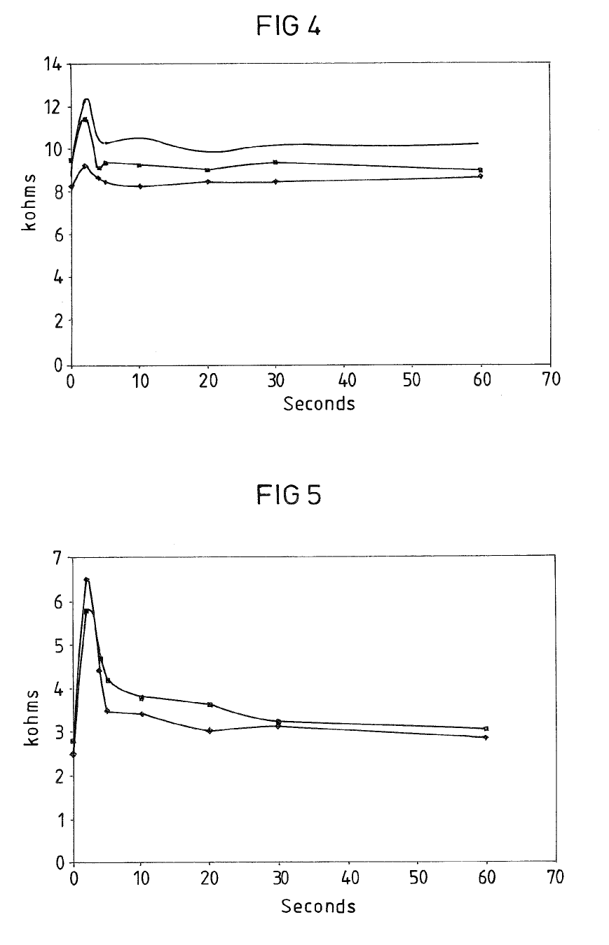

If during insertion the distal point of cannula with the stimulation electrode 12 inadvertently penetrates a blood vessel or a lymph vessel, this results in impedance measurements which are represented in FIG. 4 by three curves. As the intravascular fluid, e.g. blood and the vessel wall have about the same impedance as the body tissue here too an impedance will be measured with a base value of between 8 and 12 kΩ. If at time 0, the D5W is injected here too we find a brief rise in impedance. As however the intravascular fluid, e.g. blood flows in the vessel, this fluid carries along the D5W and carries it from the stimulation electrode 12. The impedance therefore does not rise as much with the injection of D5W and in particular falls very quickly, i.e. within about 2 seconds back to the base value.

If during spinal anesthesia the distal point of the cannula 10 with the stimulation electrode 12 penetrates inadvertently and undesired into the intrathecal area, the situation shown in FIG. 5 is created. Since the cerebrospinal fluid (CSF) in the intrathecal area has a very high electrical conductivity the measured total impedance decreases to a base value of ca. 3 to 6 kΩ when the stimulation electrode 12 is located in the intrathecal area. If D5W is injected at time 0, this initially protects the stimulation electrode 12 so that the impedance rapidly rises to values of between ca. 6 to 7 kΩ. However the D5W then distributes in the CSF so that the impedance returns to the base value of 3 to 4 kΩ. Since the CSF does not flow in the intrathecal area, the dissemination and dilution of the D5W is slower than for the intravascular injection of Figure. The impedance therefore returns to the base value in a period of about 10 seconds.

By injecting D5W the impedance values and impedance times after the injection of D5W additional information is generated which enables the localization of the cannula points. The inventive procedure therefore makes possible a localization of the position of the cannula for the nerve block using objective measurements. This results in more dependable and secure positioning of the cannulae and therefore nerve block technology. The nerve block can also be performed by a less practiced anesthetist without risk of nerve damage.

1. A device to position a cannula or a catheter for nerve block for use with a unipolar cannula (10), which is electrically insulated around its axial length, the device comprising:
a stimulation device (20) to generate an electrical signal;
an electrically conductive bare stimulation electrode (12) provided on a distal end of the unipolar cannula (10);
a skin electrode (14), which can be applied to a body surface (26) of a patient with an electrically conductive contact;
wherein the stimulation electrode (12) and the skin electrode (14) can be connected to the stimulation device (20), so that the electrical signals are routed from the stimulation electrode (12) through a patient's body (28) to the skin electrode (14);
wherein the stimulation device (20) comprises a measuring device (22) to measure the electrical resistance between the stimulation electrode (12) and the skin electrode (14), and
wherein the stimulation device (20) comprises an alarm device (24) which produces an audible alarm signal when the measured resistance exceeds an adjustable threshold value.

2. The device according to claim 1, wherein the alarm device (24) is designed to produce an acoustic signal.

3. The device according to claim 1, wherein a catheter can be introduced through the cannula (10).

4. The device according to claim 3, wherein a stimulation electrode is arranged on the distal end of the catheter to which the stimulation device (20) is connected.

5. The device according to claim 1, wherein the electrical signals are electric pulses.

6. The device according to claim 5, wherein the electric pulses are adjustable as to amplitude and/or length and/or frequency.

7. A method to position a cannula or a catheter for nerve block, comprising the following steps:
providing of a stimulation device which generates electrical signals;
providing a cannula which is electrically insulated along its axial length and is provided with a bare electrically conductive stimulation electrode on its distal end;
applying a skin electrode to the body surface of a patient in a fashion that the skin electrode is in electrically conductive contact with the body surface;
connecting the cannula and the skin electrode in electrical conductance to the stimulation device in a fashion that the electrical signals of the stimulation device are applied to the stimulation electrode of the cannula and the skin electrode;
penetrating the patient's body with the cannula;
measuring of the cannula in the patient's body between the stimulation electrode and the skin electrode when the cannula penetrates;
localizing the position of the distal end of the cannula with the stimulation electrode with respect to a nerve to be blocked based on the increase of the resistance when the stimulation electrode penetrates the nerve sheath of the nerve.

8. The method according to claim 7, wherein an alarm signal is produced when the measured resistance exceeds a threshold value.

9. The method according to claim 8, wherein the threshold value is adjustable.

10. The method according to claim 7, wherein the electrical signals are current pulses.

11. The method according to claim 10, wherein the current pulses are adjustable in terms of amplitude and/or length and/or frequency.

12. The method according to claim 7, wherein a catheter is introduced through the cannula when the distal end of the cannula is positioned on the nerve.

13. The method according to claim 12, wherein a stimulation electrode is provided on the distal end of the catheter and wherein the stimulation electrode of the catheter is connected to the stimulation device.

14. The method according to claim 7, wherein when piercing and positioning the cannula, this cannula injects a physiological fluid with low electrical conductivity.

15. The method according to claim 14, wherein the physiological fluid is D5W (5% dextrose in water).

16. The method according to claim 14, wherein the position of the point of the cannula can be localized by the increase and time of the measured resistance after injection of the fluid.

17. The method according to claim 8, wherein the alarm signal is an optical and/or preferably acoustic alarm signal
